(19) United States
(12) Patent Application Publication
(10) Pub. No.: US 2021/0085748 A1
(43) Pub. Date: Mar. 25, 2021

(54) Implantable medical device having a negatively-charged and antimicrobial surface

(71) Applicant: Peter HILDEBRANDT, Herzogenaurach (DE)

(72) Inventor: Peter HILDEBRANDT, Herzogenaurach (DE)

(21) Appl. No.: 17/024,343

(22) Filed: Sep. 17, 2020

(30) Foreign Application Priority Data
Sep. 20, 2019 (DE) 10 2019 006 638.3

(51) Int. Cl. A61K 38/08; A61K 47/12; A61K 31/727; A61K 31/65; A61L 27/28; A61L 27/54

(52) U.S. Cl. CPC A61K 38/08; A61K 47/12; A61K 31/727; A61K 31/65; A61L 2300/404; A61L 27/54; A61L 2300/236; A61L 2300/25; A61L 2300/216; A61L 27/28

(57) ABSTRACT

A completely or partially implantable medical device, the surface of which has attached to it a substance having a permanent negative charge excess, to which is in turn attached an antimicrobial substance. This imparts antimicrobial properties and at the same time repels the cellular constituents of killed bacteria.

### CROSS-REFERENCES TO RELATED APPLICATIONS

This application claims the priority of German Patent Application, Serial No. 10 2019 006 638.3, filed Sep. 20, 2019, the content of which is incorporated herein by reference in its entirety as if fully set forth herein.

### FIELD OF THE INVENTION

The invention relates to a completely or partially implantable medical device having antimicrobial properties that at the same time repels the cellular constituents of killed bacteria.

### BACKGROUND OF THE INVENTION

The background to the invention can be explained as follows. The colonization of implantable medical devices by bacteria, biofilm, and incrustations and the problems in patient care that result therefrom have been an object of research for many years. Examples of such implantable medical devices include stents, catheters, mesh implants, breast implants and cardiac pacemakers, there being a large number of further examples. Not only can bacterial colonization result in biofilm formation and infections, it can also cause other effects such as the occlusion of tubular implants by deposits and incrustation. Attaching pharmacological active substances to the surface of implantable medical devices has met with some success in this connection. The prior art thus includes numerous documents that propose, for example, a covalent attachment of antibiotic active substances to the surface, as described for example in US 2011/135703 A1 and AU 2013245551 B2.

Many antibiotic active substances are however affected by bacterial resistance. This is caused by bacterial strains developing mutations that result in the antibiotic active substances no longer having any effect on them. A current focus of research is peptides that have antimicrobial properties, since problems with resistance are thus far not a problem here. For example, CA2655168 discloses the covalent attachment of peptides having antimicrobial properties via intermediate molecules to produce an antimicrobial surface. Studies have however shown that the antimicrobial properties of such coatings are not permanent. This can be explained as follows: Antimicrobial peptides and many antibiotics have a positive charge excess due to their numerous amino groups. This results in electrostatic attraction of bacteria, which have a negative charge excess. In the case of Gram-negative bacteria, the negative charge of the bacteria arises from the strongly negatively charged lipopolysaccharides in the cell wall, in the case of Gram-positive bacteria, it arises from the negatively charged phosphate group of the phosphodiester bonds between the teichoic acid monomers in the cell wall. Although the bacteria are killed by the antimicrobial action of the peptide coating, the cellular constituents of these bacteria remain adhering to the surface. Over time, this results in them forming a layer that envelops the antimicrobial surface, thereby preventing the coating from exerting its action on newly arriving bacteria. The consequence of this is that such coatings have no long-term effectiveness.

A different approach is disclosed in patent EP 0 890 367 B1: the covalent attachment of glycosaminoglycans to a urological implant. As later research studies have shown, this approach has indeed proved successful.

The negative charge of the glycosaminoglycans is regarded as a critical factor for the effectiveness (Int J Antimicrob Agents. 2004, 23: Bacterial biofilm formation on urologic devices and heparin coating as preventive strategy. Tenke P. et al.). Since, as already mentioned, bacteria have a negative charge excess too, this results in electrostatic repulsion from the surface coated with glycosaminoglycans. Heparin is the glycosaminoglycan with the highest negative charge here.

However, glycosaminoglycans do not have any antimicrobial properties, which means that a coating thereof is unable to kill bacteria. The same applies to the negatively charged substances for the repulsion of bacteria proposed in German application 102018214299.8.

Against this background, the prior art can be seen to be in need of improvement.

### SUMMARY OF THE INVENTION

To address the described problems of the prior art, the invention proposes as a basic concept the attachment to the surface of a substance having a permanent negative charge excess in order to achieve bacteria-repellent properties. In order that persisting antimicrobial properties are obtained, an antimicrobial substance is attached to this negatively charged substance. This addresses the previously described problem of killed bacteria adhering to the surface.

There are various options for achieving the negatively charged substance. What is absolutely fundamental here is ensuring that this substance permanently retains its negative charge excess on the surface and that its negative charge is not neutralized by chemical reactions in the region of the implant. Such reactions can occur, for example, when there is contact with tissue and body fluids such as blood, urine or wound secretions.

For such a basic substance, there are various advantageous variants. The invention proposes the attachment for this purpose of one or more different substances that contain carboxylic acid, sulfonic acid or phosphonic acid groups or combinations thereof. At the pH prevailing in the human body, these groups are deprotonated and thus negatively charged.

When selecting the negatively charged substance, it is also important that it is possible to attach an antimicrobial substance to it. This can be done for example via free carboxyl or amino groups. According to the invention, there must be at least two such groups present: one in order to be able to attach the substance to the surface and a second one in order to be able to attach an antimicrobial substance to the first substance. Accordingly the implantable medical device comprises a surface-attached substance having a permanent negative charge excess, wherein the negative charges are borne by one of carbonic acid groups, sulfonic acid groups, phosphonic acid groups and combinations thereof and this substance additionally has at least two molecular groups that are one of carboxyl groups, amino groups and a combination thereof, and by an antimicrobial substance that is linked to the substance having a negative charge excess. According to a preferred embodiment at least three such mentioned groups are present. For example, mellitic acid, which has six carboxyl groups, and heparin, which has multiple carboxyl and amino groups, are accordingly cited as preferred embodiments.

A medical device, in which the substance having the permanent negative charge excess is applied with comprehensive coverage, but that the antimicrobial substance is applied to the surface with only partial coverage, allows for the possibility that the attachment of the antimicrobial substance is not comprehensive in its coverage. By contrast, the prior attachment of the negatively charged substance is carried out with comprehensive coverage, which means that an overall negative charge excess persists. This can be advantageous particularly when an antimicrobial substance having a positive charge excess is used. It can be achieved, as described in exemplary embodiment II, by incomplete activation of the attachment sites of the negatively charged substance that was applied first. This means that not all of the attachment sites on the surface are available for attachment of the antimicrobial substance, with the result that the layer of the antimicrobial substance that forms on the comprehensively covering layer of the negatively charged substance is not comprehensive in its coverage and contains gaps.

Suitable antimicrobial substances are, for example, for the reasons mentioned above, antimicrobial peptides according to the invention, such as are disclosed in US2018193411, the peptide of the sequence kwivwrwrfkr-NH₂ having been found to be particularly effective here, this being specified in the invention. However, broad-spectrum antibiotic substances such as aminopenicillins or tetracyclines are likewise suitable and are proposed in the invention. All the antimicrobial substances mentioned here have carboxyl or amino groups, or both, which permit attachment to amino or carboxyl groups of the substance having a negative charge excess via peptide bonds. Attachment to the surface of the medical device via peptide bonds has long-term stability. Other linkages are however also possible, for example an ionic bond.

### DESCRIPTION OF PREFERRED EMBODIMENTS

The invention is elucidated in more detail hereinbelow with reference to following various exemplary embodiments:

### Example I—Covalent Coating Process with Mellitic Acid and Antimicrobial Peptide on a Medical Implant Having a Polyurethane Surface

1) The polyurethane surface is immersed in an ether solution of hexamethylene diisocyanate for 12 h to prepare it for attachment of mellitic acid, after which it is rinsed in deionized water.
2) The thus prepared polyurethane surface is then immersed in an aqueous solution of sodium hydrogen carbonate for 12 h for hydrolysis, after which it is rinsed in deionized water.
3) The thus prepared polyurethane surface is immersed in an aqueous solution of mellitic acid and N-(3-dimethylaminopropyl)-N′-ethylcarbodiimide for 12 h to attach the mellitic acid via a peptide bond, after which it is rinsed in deionized water.
4) The thus prepared polyurethane surface is immersed in an aqueous solution of N-(3-dimethylaminopropyl)-N′-ethylcarbodiimide for 1 h to prepare it for the attachment of the peptide.
5) To attach the antimicrobial peptide, the thus prepared polyurethane surface is immersed in an aqueous solution containing the peptide having the sequence kwivwrwrfkr-NH2 for 12 h, after which it is rinsed in deionized water.

### Example II—Covalent Coating Process with Heparin and Doxycycline on a Medical Implant Having a Silicone Surface

1) The silicone surface is immersed in an aqueous solution of 3-aminopropyltriethoxysilane for 12 h to prepare it for the attachment of heparin, after which it is rinsed in deionized water.
2) The thus prepared silicone surface is immersed in an aqueous solution of adipic acid and N-(3-dimethylaminopropyl)-N′-ethylcarbodiimide for 12 h to attach the heparin via a peptide bond, after which it is rinsed in deionized water.
3) The thus prepared silicone surface is immersed in an aqueous solution of N-(3-dimethylaminopropyl)-N′-ethylcarbodiimide for 1 h to prepare it for the attachment of the heparin.
4) To attach the heparin via a peptide bond, the thus prepared silicone surface is immersed in an aqueous solution containing heparin for 12 h, after which it is rinsed in deionized water.
5) The thus prepared silicone surface is immersed in an aqueous solution of N-(3-dimethylaminopropyl)-N′-ethylcarbodiimide for 1 h to prepare it for the attachment of the doxycycline.
As a variant, it is possible to only partially activate the carboxyl groups of heparin, thereby achieving non-comprehensive coverage by the doxycycline. This is the case when there are fewer carbodiimide molecules in the solution than carboxyl groups on the surface or when the immersion time is reduced or when both measures are combined.
6) To attach the doxycycline via a peptide bond, the thus prepared silicone surface is immersed in an aqueous solution containing doxycycline for 12 h, after which it is rinsed in deionized water.

Accordingly furnished silicone samples have proved very successful in microbiological tests. Compared to uncoated silicone samples, the number of living bacteria on the surface was markedly reduced after immersion in bacterial suspensions for 24 hours. Compared to samples furnished exclusively with antimicrobial substances, the number of killed bacteria on the surface was drastically reduced, which is crucial to the maintenance of effectiveness when exposed to bacteria for long periods.

What is claimed is:

1. An implantable medical device comprising a surface-attached substance having a permanent negative charge excess, wherein the negative charges are borne by one of carbonic acid groups, sulfonic acid groups, phosphonic acid groups and combinations thereof and this substance additionally has at least two molecular groups that are one of carboxyl groups, amino groups and a combination thereof, and by an antimicrobial substance that is linked to the substance having a negative charge excess.

2. The medical device as claimed in claim 1, wherein the surface-attached substance additionally has at least three molecular groups that are one of carboxyl groups, amino groups and a combination thereof

3. The medical device as claimed in claim 1, wherein the employed substance having a permanent negative charge excess is mellitic acid.

4. The medical device as claimed in claim 1, wherein the employed substance having a permanent negative charge excess is heparin.

5. The medical device as claimed in claim 1, wherein the substance having the permanent negative charge excess is applied with comprehensive coverage, but that the antimicrobial substance is applied to the surface with only partial coverage.

6. The medical device as claimed in claim 1, wherein the employed antimicrobial substance is an antimicrobial peptide.

7. The medical device as claimed in claim 6, wherein the employed antimicrobial substance is an antimicrobial peptide having the sequence kwivwrwrfkr-NH2.

8. The medical device as claimed in claim 1, wherein the employed antimicrobial substance is an antibiotic.

9. The medical device as claimed in claim 8, wherein the employed antibiotic is one of an aminopenicillin and a tetracycline.

10. The medical device as claimed in claim 1, wherein the substances are attached to the surface of the medical device by means of peptide bonds.

11. The medical device as claimed in claim 1, wherein the medical device is completely implantable.

12. The medical device as claimed in claim 1, wherein the medical device is partially implantable.
